(19) United States
(12) Patent Application Publication
(10) Pub. No.: US 2009/0272383 A1
(43) Pub. Date: Nov. 5, 2009

(54) Contraceptive Device

(76) Inventor: Jerrold A. Wish, Miami Beach, FL (US)

Correspondence Address: O'BRIEN JONES, PLLC, 8200 Greensboro Drive, Suite 1020A, McLean, VA, 22102, US

(21) Appl. No.: 12/115,376

(22) Filed: May 5, 2008

(51) Int. Cl. A61F 6/04

(52) U.S. Cl. 128/844

(57) ABSTRACT

A contraceptive device is disclosed. The contraceptive device comprises a tubular sheath region having a contiguous sheath and at least one sensitizing area. A leak-proof tip region is located at one end of the tubular sheath and an open tubular end is located at the other end of the tubular sheath region. The thickness of the at least one sensitizing area is less than the thickness of the contiguous sheath. The tubular end may have at least one tab to aid in removal of the contraceptive device.

### FIELD

The present invention relates generally to a contraceptive device, and more particularly to a contraceptive device that increases tactile sensation.

### INTRODUCTION

The recent significant increase in the incidence and spread of sexually transmitted diseases (STD's) has resulted in increased use of condoms as a prophylactic measure to reduce the risk of infection and transmission of STD's.

Condoms generally comprise elongated thin, flexible, tubular sheaths made of a resilient, rubber-like material, such as rubber (e.g., latex) or various thermoplastic elastomers.

Condoms are regulated by the United States Food and Drug Administration (“FDA”) as Class II medical devices. Under FDA rules, a condom is a sheath that must completely cover the penis with a closely fitting membrane. See 21 C.F.R. 884.5300. In addition to condoms, the FDA rules also define another class of contraceptive devices: glans sheaths. A glans sheath covers the glans, but does not cover the entire shaft of the penis. See 21 C.F.R. 5320. Glans sheaths are used for contraceptive purposes only.

A common complaint regarding the use of condoms during sexual intercourse is the change in feeling, as compared to when no contraceptive is used, or diminishment of tactile sensation.

To improve the feel of condoms, some manufacturers have reduced the thickness of the material used to make the condom. Condoms made with reduced thickness material generally have a uniform material thickness throughout the entire condom. This reduction in condom thickness, however, may raise concerns of tearing, puncturing, or otherwise breaching the condom, which may allow the transmittal of fluids that increase the risk of transmitting sexually transmitted diseases or increase the chances of an unplanned pregnancy.

### SUMMARY

The present invention may address one or more of the above-mentioned issues. Other features and/or advantages may become apparent from the description which follows.

Certain embodiments of the present teachings provide a contraceptive device comprising a tubular sheath region comprising a contiguous sheath and at least one sensitizing area, a leak-proof tip region at one end of the tubular sheath, and an open tubular end at the other end of the tubular sheath. The contiguous sheath has a thickness, T, and the at least one sensitizing area has a thickness, t, wherein t<T.

Certain embodiments of the present teachings alternatively or additionally provide a contraceptive device comprising a tubular sheath region comprising a contiguous sheath and at least one first sensitizing area, a leak-proof tip region at one end of the tubular sheath, and an open tubular end at the other end of the tubular sheath. The contiguous sheath has a thickness, T, and the at least one first sensitizing area has a thickness t₁ that is different than the thickness T of the contiguous sheath.

Certain embodiments of the present teachings may also provide a contraceptive device comprising a tubular sheath region comprising a contiguous sheath having a thickness T, at least one first sensitizing area having a thickness t₁, and at least one second sensitizing area having a thickness t₂, wherein t₁<T and t₂<T.

In the following description, certain aspects and embodiments will become evident. It should be understood that the invention, in its broadest sense, could be practiced without having one or more features of these aspects and embodiments. It should be understood that these aspects and embodiments are merely exemplary and explanatory and are not restrictive of the invention.

### BRIEF DESCRIPTION OF THE DRAWINGS

Features and advantages of the claimed subject matter will be apparent from the following detailed description of embodiments consistent therewith, which description should be considered with reference to the accompanying, wherein:

FIG. 1 is a side view of a contraceptive device according to an embodiment of the present teachings;

FIG. 2 is a cutaway partial sectional view of the sheath region according to the embodiment of FIG. 1, taken along line 2-2 of FIG. 1;

FIG. 3 is a cutaway partial sectional view of a sheath region of a contraceptive device according to another embodiment of the present teachings;

FIG. 4 is a cutaway partial sectional view of a sheath region of a contraceptive device according to yet another embodiment of the present teachings;

FIG. 5 is a side view of a contraceptive device according to another embodiment of the present teachings;

FIG. 6 is a side view of a contraceptive device according to another embodiment of the present teachings; and

FIG. 7 is a side view of a contraceptive device according to another embodiment of the present teachings.

Although the following detailed description makes reference to illustrative embodiments, many alternatives, modifications, and variations thereof will be apparent to those skilled in the art. Accordingly, it is intended that the claimed subject matter be viewed broadly.

### DETAILED DESCRIPTION OF VARIOUS EMBODIMENTS

Reference will now be made to various embodiments, examples of which are illustrated in the accompanying drawings. However, these various exemplary embodiments are not intended to limit the disclosure. To the contrary, the disclosure is intended to cover alternatives, modifications, and equivalents.

The present teachings contemplate embodiments for contraceptive devices including areas within the sheath region having a thickness less than the contraceptive material thickness in the tip region, which can include a thickness of zero. In the context of the present disclosure, the areas within the sheath region having a thickness less than the thickness in the tip region are referred to as “sensitizing areas”.

The contraceptive device in accordance with the present teachings may be of any known material and formed via any method known in the art, including molding, extrusion, heat sealing, or dipping. The sensitizing areas according to the present teachings may be formed via any method known in the art, including punching, cutting, or molding. Non-zero thickness sensitizing areas according to the present teachings may be formed by any known method in the art, including molding, pressing, stamping, or cutting.

In the context of the present teachings, the term “sensitizing area” means an area on the contraceptive device having a thickness less than the thickness of the material in the tip of the contraceptive device. The thickness of the sensitizing area includes areas of zero thickness.

In the context of the present teachings, the term “contiguous” means an unbroken and continuous span of material. For example, a contiguous sheath according to the present teachings comprises material that is substantially unbroken as it extends from the tip region to the tubular end and around the sensitizing areas. A contiguous sheath according to the present teachings may resemble a web of material.

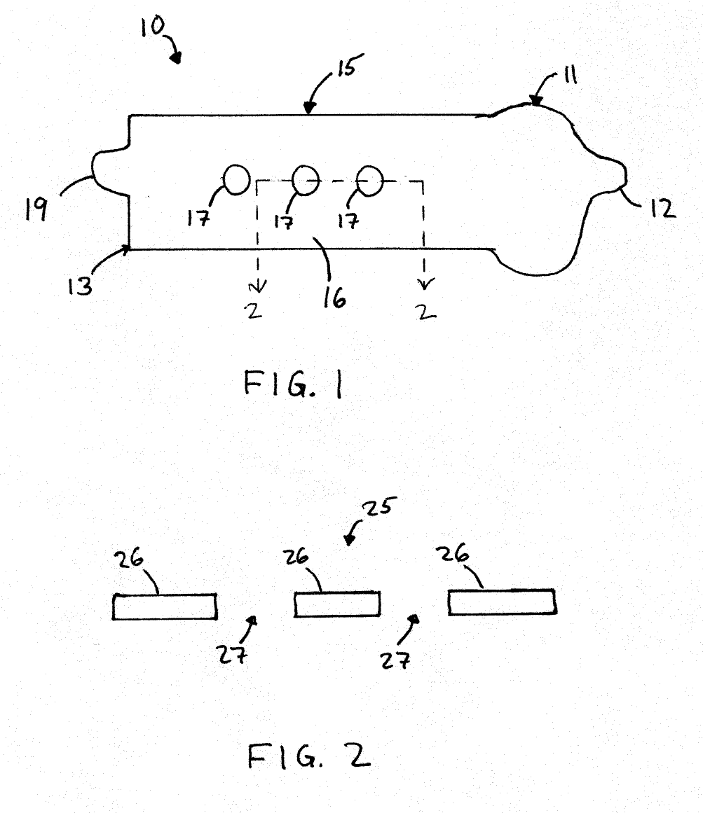

In accordance with certain embodiments of the present teachings, contraceptive device 10 as illustrated in FIG. 1 comprises a selected thermoplastic elastomer material, such as a polyester-based polyurethane or polyurethane elastomer (e.g. ELASTOLLAN®). Contraceptive device 10 comprises a closed tip region 11, a tubular sheath region 15, and an open tubular end 13 opposite tip region 11. Tip region 11 is unbroken and leak-proof. Tip region 11 comprises a reservoir 12 to contain ejaculate. In certain embodiments of the present teachings, the material of tip region 11 may have a thickness, for example, ranging from about 0.03 mm to about 0.15 mm. In at least one embodiment of the present teachings, tip region 11 has a thickness ranging from about 0.05 mm to about 0.08 mm.

Sheath region 15 of contraceptive device 10 extends from the tip region 11 to the tubular end 13 and is formed of at least two regions having different thicknesses, sheath 16 and sensitizing areas 17. Sheath 16 is contiguous between tip region 11 and tubular end 13 and can be similar in thickness to tip region 11. According to the present disclosure, the term “contiguous” means that at least one continuous path can be traced between tip region 11 and tubular end 13 so that the material of the sheath 16 is substantially unbroken as it extends around the sensitizing areas 17.

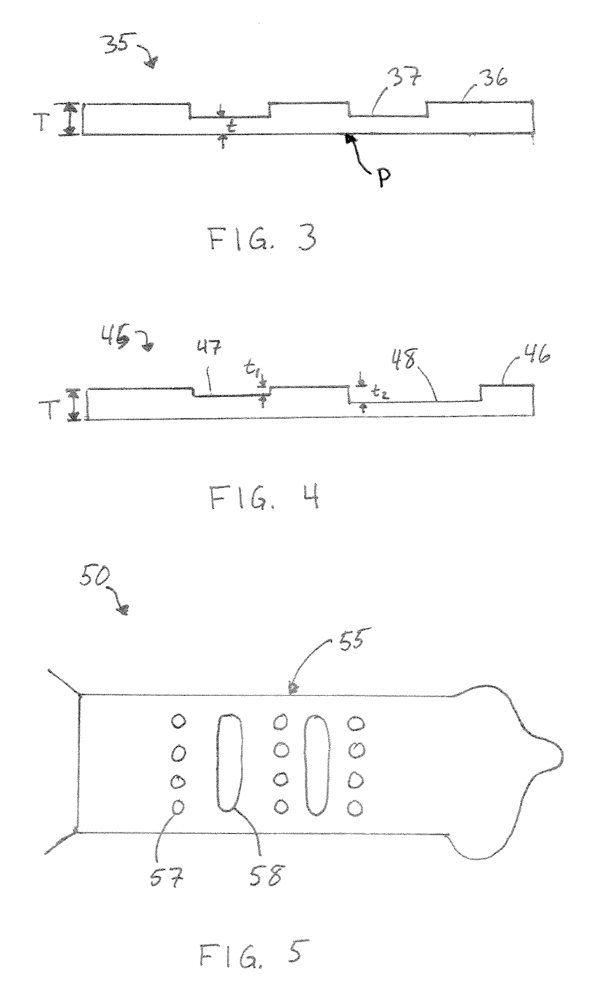

As shown in FIGS. 2 and 3, the sensitizing areas may have a variety of thicknesses. In FIG. 2, sheath region 25 comprises sensitizing areas 27 having a thickness of zero and sheath 26 has a thickness, T. FIG. 3 depicts sheath region 35 comprising sensitizing areas 37 having a thickness, t, which is greater than zero and less than the thickness, T, of sheath 36. As shown in FIG. 4, the sheath region 45 may have a combination of at least two different sensitizing areas 47, 48 having different thicknesses, t₁ and t₂, respectively, each of which are less than the thickness, T, of sheath 46. Although areas with thickness T, t₁, t₂ may not provide as great a sensation, they can make a device more leak-proof.

The position of the sensitizing areas in relation to the sheath may also vary. In FIG. 3, sheath 36 and sensitizing areas 37 form a planar surface P on one side of sheath region 35. Planar surface P may be on either the interior surface or exterior surface of sheath region 35. Alternatively, the sensitizing areas and the sheath region may not form a planar surface on either the interior or exterior surface of the sheath region.

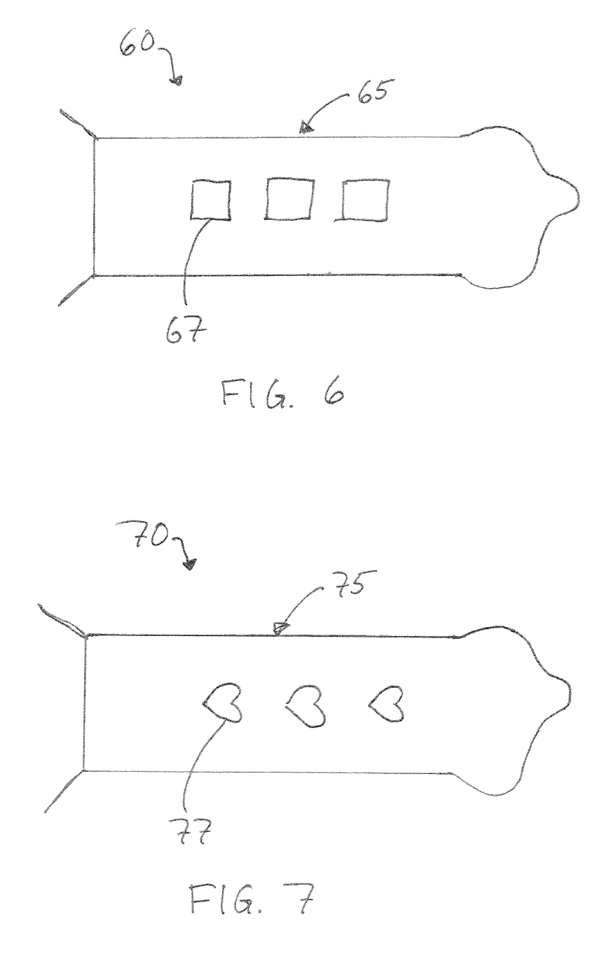

Sensitizing areas may comprise any shape. In FIG. 1, sensitizing areas 17 are circular. Other examples of sensitizing area shapes are shown in FIGS. 5-7 which are intended to convey that the sensitizing areas can have any shape or design. Contraceptive device 50, illustrated in FIG. 5, comprises sheath region 55 having first sensitizing areas 57 and second sensitizing areas 58. First sensitizing areas 57 are circular and second sensitizing areas 58 are oblong or oval in shape. FIG. 6 depicts contraceptive device 60 having a sheath region 65 with square sensitizing areas 67. In FIG. 7, heart-shaped sensitizing areas 77 are formed within sheath region 75 of contraceptive device 70.

The contraceptive device may comprise as few as 1 or 2 sensitizing areas or as many as 1000 or more. In at least one embodiment, the contraceptive device comprises from about 3 to about 100 sensitizing areas. In certain embodiments of the present teachings the sensitizing areas cover less than half of the total area of the sheath region. In at least one embodiment of the present teachings, the sensitizing areas cover less than a quarter of the total area of the sheath region.

Tubular end 13 of contraceptive device 10 may comprise at least one tab 19. Tab 19 extends from tubular end 13 to aid in the removal of contraceptive device 10. In at least one embodiment, contraceptive device 10 comprises from 1 to 3 tabs 19. The at least one tab 19 can have a variety of shapes and sizes selected for ease of use and aesthetic appeal, and/or based on manufacturing and packaging concerns. For example, as shown, the tabs can be substantially rectangular. The tabs could alternatively comprise, for example, a heart shape or other decorative design.

Other embodiments of the disclosure will be apparent to those skilled in the art from consideration of the specification and practice of the teachings disclosed herein.

In certain embodiments of the present teachings, a lubricant or other complimentary liquid may be applied to the contraceptive device. Complimentary liquids may include, for example, spermicides, flavorings, aromatic liquids, warming liquids, etc.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the written description and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “an adhesive” can include two or more different adhesives. As used herein the term “include” and its grammatical variants are intended to be non-limiting, such that recitation of items in a list is not to the exclusion of other like items that can be substituted or added to the listed items.

It will be apparent to those skilled in the art that various modifications and variation can be made to the sample preparation device and method of the present disclosure without departing from the scope its teachings. Other embodiments of the disclosure will be apparent to those skilled in the art from consideration of the specification and practice of the teachings disclosed herein. It is intended that the specification and examples be considered as exemplary only.

What is claimed is:

1. A contraceptive device comprising:
a tubular sheath region comprising a contiguous sheath and at least one sensitizing area;
a leak-proof tip region at one end of the tubular sheath; and
an open tubular end at the other end of the tubular sheath;
wherein the contiguous sheath has a thickness, T, and the at least one sensitizing area has a thickness, t, wherein t<T.

2. The contraceptive device of claim 1, wherein the thickness of the contiguous sheath ranges from about 0.03 mm to about 0.15 mm.

3. The contraceptive device of claim 2, wherein the thickness of the contiguous sheath ranges from about 0.05 mm to about 0.08 mm.

4. The contraceptive device of claim 1, further comprising at least one tab at the tubular end.

5. The contraceptive device of claim 1, wherein t=0.

6. The contraceptive device of claim 1, wherein the at least one sensitizing areas and the contiguous sheath form a planar surface on an interior surface of the sheath region.

7. The contraceptive device of claim 1, wherein the at least one sensitizing areas and the contiguous sheath form a planar surface on an exterior surface of the sheath region.

8. A contraceptive device comprising:
a tubular sheath region comprising a contiguous sheath and at least one first sensitizing area;
a leak-proof tip region at one end of the tubular sheath; and
an open tubular end at the other end of the tubular sheath;
wherein the contiguous sheath has a thickness, T, and the at least one first sensitizing area has a thickness t₁ that is different than the thickness T of the contiguous sheath.

9. The contraceptive device of claim 8, wherein the thickness of the contiguous sheath ranges from about 0.03 mm to about 0.15 mm.

10. The contraceptive device of claim 9, wherein the thickness of the contiguous sheath ranges from about 0.05 mm to about 0.08 mm.

11. The contraceptive device of claim 8, further comprising at least one second sensitizing area having a thickness, t₂, wherein t₂<T.

12. The contraceptive device of claim 11, wherein thicknesses t₁ and t₂ are different and are less than T.

13. The contraceptive device of claim 11, wherein the at least one first sensitizing area and the at least one second sensitizing area comprise similar shapes.

14. The contraceptive device of claim 11, wherein the at least one first sensitizing area and the at least one second sensitizing area comprise different shapes.

15. A contraceptive device comprising:
a tubular sheath region comprising a contiguous sheath having a thickness T, at least one first sensitizing area having a thickness t₁, and at least one second sensitizing area having a thickness t₂, wherein t₁<T and t₂<T.

16. The contraceptive device of claim 15, wherein the thickness of the contiguous sheath ranges from about 0.03 mm to about 0.15 mm.

17. The contraceptive device of claim 16, wherein thickness of the contiguous sheath ranges from about 0.05 mm to about 0.08 mm.

18. The contraceptive device of claim 15, wherein the at least one first sensitizing area and the at least one second sensitizing area comprise similar shapes.

19. The contraceptive device of claim 15, wherein the least one first sensitizing area and the at least one second sensitizing area comprise different shapes.

20. The contraceptive device of claim 15, wherein t₁≠t₂.
